(19) United States
(12) Patent Application Publication
(10) Pub. No.: US 2007/0122443 A1
(43) Pub. Date: May 31, 2007

(54) Amphiphilic copolymer compositions

(76) Inventors: Pallassana V. Narayanan, Belle Mead, NJ (US); Jonathon Z. Zhao, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/288,760

(22) Filed: Nov. 29, 2005

(51) Int. Cl. A61K 31/785

(52) U.S. Cl. 424/423; 424/78.23; 424/78.3

(57) ABSTRACT

The present invention provides an amphiphilic coating material for applying on at least a portion of one surface of an article. The amphiphilic coating material comprises a copolymer containing one or more alkyl methacrylate or alkyl acrylate co-monomer units; one or more vinyl acetate co-monomer units; and up to 40% mole of polyethylene oxide substituted methacrylate co-monomer units. Optionally, one or more biologically active molecules may be covalently bonded to the polyethylene oxide substituted methacrylate co-monomer units. The present invention also provides an article having the inventive amphiphilic coating thereon.

### FIELD OF INVENTION

The present invention relates to an amphiphilic coating material for application to at least a portion of one surface of an article. The present invention also relates to an article having the inventive amphiphilic coating.

### BACKGROUND OF INVENTION

Most medical devices are made from metals, ceramics, or polymeric materials. However, these materials are hydrophobic, non-conformal, and non-slippery, and thereby may cause thrombus formation, inflammation, or other injuries to mucous membranes during use or operation. Thus, the issue of biocompatibility is a critical concern for manufacturers of medical devices, particularly medical implants. In order to function properly and safely, medical devices are usually coated with one or more layers of biocompatible materials. The coatings on these medical devices may, in some instances, be used to deliver therapeutic and pharmaceutical agents.

Since medical devices, particularly implantable medical devices, are intended for prolonged use and directly interface with body tissues, body fluids, electrolytes, proteins, enzymes, lipids, and other biological molecules, the coating materials for medical devices must meet stringent biological and physical requirements. These requirements, as a minimum, include the following: (1) the coatings must be hydrophilic and lubricous when in contact with body tissue, and thereby increase patient comfort during operation and enhance the maneuverability of the medical device; (2) the coatings must be flexible and elastic, so they conform to the biological structure without inducing detrimental stress; (3) the coatings must be hemocompatible, and thereby reduce or avoid formation of thrombus or emboli; (4) the coatings must be chemically inert to body tissue and body fluids; and (5) the coatings must be mechanically durable and not crack when formed on medical devices. If the coatings are impregnated with pharmaceutical or therapeutic agents, it is typically required that the coatings and the formation thereof are compatible with the pharmaceutical or therapeutic agents. If the coatings are used as coatings and the underlying basecoats are impregnated with pharmaceutical or therapeutic agents, it is further required that the coating and the formation thereof must be compatible with the basecoat and the pharmaceutical or therapeutic agents impregnated therein; and the coating must allow the pharmaceutical or therapeutic agents to permeate therethrough. It is also desirable that the coating functions as a physical barrier, a chemical barrier, or a combination thereof to control the elution of the pharmaceutical or therapeutic agents in the underlying basecoat.

In order to combine the desired properties of different polymeric materials, the conventional coating composition for commercial drug eluting stents used a polymer blend, i.e., physical mixture, of poly ethylene-vinyl acetate (EVAc) and poly butyl methacrylate (BMA). However, one disadvantage of this conventional coating is the phase separation of the polymer blend, which can be detrimental to the performance of the coating and the stability of drugs impregnated therein.

Another coating composition of the prior art comprises a supporting polymer and a hydrophilic polymer, wherein the supporting polymer contains functional moieties capable of undergoing crosslinking reactions and the hydrophilic polymer is associated with the supporting polymer (see, for example, U.S. Pat. No. 6,238,799). However, the preparation of this prior art coating composition employs chemical crosslinking reactions and a high temperature curing process, which are not compatible with a drug-containing coating.

The prior art also uses a coating composition formed by the gas phase or plasma polymerization of a gas comprising monomers of polyethylene glycol vinyl ether compounds (see, for example, U.S. Patent Application Publication 2003/0113477). However, the polymer prepared through the plasma process has poorly defined molecular weight and a large polydispersity. The plasma laid polymers of low molecular weight have limited mechanical durability. Further, plasma treatment can penetrate through the underlying basecoat and damage the drug content therein. Another problem with this prior art approach is that the free radicals or other high energy species generated in the plasma process may persist in the coating and cause drug content loss in the basecoat over time.

To decrease thrombosis caused by the use of medical devices, the prior art modifies the coatings of medical devices via conjugating, i.e., covalently bonding, an antithrombotic agent to the coatings (see, for example, U.S. Pat. No. 4,973,493 and www.surmodics.com). Although this approach may produce a coating with excellent antithrombotic property, the prior art conjugation methods employ UV-radiating processes and/or chemical crosslinking processes, which may cause degradation of the antithrombotic agent in the coating.

Thus, there remains a need for a coating material that can satisfy the stringent requirements, as described above, for applying on at least one surface of a medical device and can be prepared through a process that is compatible with the sensitive pharmaceutical or therapeutic agents impregnated in the coatings.

### SUMMARY OF THE INVENTION

Accordingly, the present invention provides an amphiphilic coating material for applying on at least a portion of one surface of an article. By “amphiphilic”, it is meant having the property of hydrophobicity and hydrophilicity simultaneously. The amphiphilic coating material comprises a copolymer containing one or more alkyl methacrylate or alkyl acrylate co-monomer units; one or more vinyl acetate co-monomer units; and up to 40% mole of polyethylene oxide substituted methacrylate co-monomer units. Optionally, one or more biologically active molecules may be covalently bonded to the polyethylene oxide substituted methacrylate co-monomer units.

Preferably, the polyethylene oxide substituted methacrylate co-monomer unit comprises the following structure: 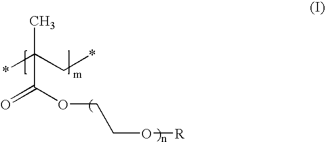
wherein R is a hydrogen atom, an alkyl group of 1 to 6 carbon atoms, or a biologically active molecule; n is an integer of 2 to 100; and m is an integer of 100 to 5000.

The present invention also provides an article having an amphiphilic coating thereon. The amphiphilic coating comprises a copolymer containing one or more alkyl methacrylate or alkyl acrylate co-monomer units; one or more vinyl acetate co-monomer units; and up to 40% mole of polyethylene oxide substituted methacrylate co-monomer units. Optionally, one or more biologically active molecules may be covalently bonded to the polyethylene oxide substituted methacrylate co-monomer units. Preferably, the article is a medical device or a component of a medical device.

### DETAILED DESCRIPTION OF THE INVENTION

The present invention provides an amphiphilic coating material for applying on at least a portion of one surface of an article. The amphiphilic coating material comprises a copolymer containing one or more alkyl methacrylate or alkyl acrylate co-monomer units; one or more vinyl acetate co-monomer units; and up to 40% mole of polyethylene oxide substituted methacrylate co-monomer units. The co-monomers in the copolymer, i.e., the alkyl methacrylate or alkyl acrylate co-monomers, the vinyl acetate co-monomers, and the polyethylene oxide substituted methacrylate co-monomers, are in random sequence. By “alkyl methacrylate”, it is meant a methacrylate derivative wherein the oxygen atom attached to the carbon atom of the carbonyl group is substituted with an alkyl group. By “alkyl acrylate”, it is meant an acrylate derivative wherein the oxygen atom attached to the carbon atom of the carbonyl group is substituted with an alkyl group. By “polyethylene oxide substituted methacrylate”, it is meant a methacrylate derivative wherein the oxygen atom attached to the carbon atom of the carbonyl group is substituted with a polyethylene oxide moiety.

The copolymer has a hydrophobic backbone that is formed by polymerization of vinyl groups. The polyethylene oxide moieties of the polyethylene oxide substituted methacrylate co-monomers provide hydrophilic pendent chains that are interspersed along the hydrophobic backbone. The polyethylene oxide moieties of the polyethylene oxide substituted methacrylate co-monomers also provide functional groups where one or more biologically active molecules may be attached. The “biologically active molecule” as used herein denotes a compound or substance having an effect on or eliciting a response from living tissue. The biologically active molecule is attached to the polyethylene oxide moiety via forming a covalent bond with the oxygen atom at the far end position of the polyethylene oxide moiety. By “the oxygen atom at the far end position”, it is meant the oxygen atom of the polyethylene oxide moiety that is furthest apart from the carbonyl group in the polyethylene oxide substituted methacrylate. The hydrophilic pendent chains of the copolymer swell under the physiological condition and form a flexible and lubricious three-dimensional network, thereby providing a hydrophilic environment for retaining the optimal activity of the attached biologically active molecules.

Preferably, the polyethylene oxide substituted methacrylate co-monomer unit comprises the following structure: 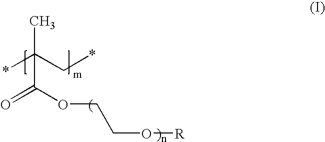
wherein R is a hydrogen atom, an alkyl group having 1 to 6 carbon atoms, or a biologically active molecule; n is an integer of 2 to 100; and m is an integer of 100 to 5000. Preferably, n is an integer of 2 to 10. The alkyl group suitable for the present invention may be straight, branched, or cyclic. Examples of suitable alkyl groups include, but are not limited to: methyl, ethyl, propyl, isopropyl, butyl, isobutyl, tert-butyl, n-pentyl, cyclopropyl, cyclobutyl, and cyclopentyl. Preferably, the alkyl group is methyl.

The biologically active molecules are covalently bonded to the polyethylene oxide moieties through conjugation processes. The conjugation process may involve one or more chemical or photo radiation reactions. The biologically active molecules suitable for the present invention include, for example, any drugs, agents, compounds and/or combination thereof that have therapeutic effects for treating or preventing a disease or a biological organism's reaction to the introduction of the medical device to the organism. Preferred biological active molecules include, but are not limited to: anti-thrombogenic agents, immuno-suppressants, anti-neoplastic agents, anti-inflammatory agents, angiogenesis inhibitors, protein kinase inhibitors, and other agents which may cure, reduce, or prevent restenosis in a mammal. Examples of the biological active molecules of the present invention include, but are not limited to: heparin, albumin, streptokinase, tissue plasminogin activator (TPA), urokinase, rapamycin, paclitaxel, pimecrolimus, and their analogs and derivatives. When the copolymer comprises more than one biologically active molecules, the biologically active molecules can be the same or different.

Preferably, the alkyl methacrylate co-monomer unit has the following general formula: 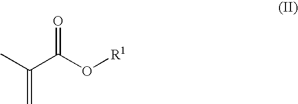
wherein R¹ is an alkyl group having 1 to 12 carbon atoms. The alkyl group may be straight, branched, or cyclic. Examples of suitable alkyl groups include, but are not limited to: methyl, ethyl, propyl, isopropyl, butyl, isobutyl, tert-butyl, n-pentyl, cyclopropyl, cyclobutyl, and cyclopentyl. Preferably, the alkyl group is methyl or butyl.

Preferably, the alkyl acrylate co-monomer unit has the following general formula: 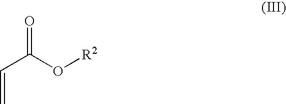
wherein R² is an alkyl group having 1 to 12 carbon atoms. The alkyl group may be straight, branched, or cyclic. Examples of suitable alkyl groups include, but are not limited to: methyl, ethyl, propyl, isopropyl, butyl, isobutyl, tert-butyl, n-pentyl, cyclopropyl, cyclobutyl, and cyclopentyl. Preferably, the alkyl group is methyl or butyl.

When the copolymer comprises more than one alkyl methacrylate or alkyl acrylate co-monomers, the more than one methacrylate or acrylate co-monomers can be the same or different. Preferred alkyl methacrylate co-monomers include methyl methacrylate, ethyl methacrylate, butyl methacrylate, and cyclic alkyl methacrylate. Preferred alkyl acrylate co-monomers include methyl acrylate, ethyl acrylate, butyl acrylate, and cyclic alkyl acrylate. It is understood to one skilled in the art that suitable alkyl methacrylate or acrylate co-monomers also include any analogous alkyl methacrylates or alkyl acrylates of the above-mentioned alkyl methacrylate and alkyl acrylate co-monomers.

Preferably, a vinyl acetate monomer has the following general formula: 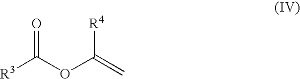
wherein R³ is an alkyl group having 1 to 6 carbon atoms; and R⁴ is a hydrogen atom or an alkyl group having 1 to 6 carbon atoms. The alkyl group suitable for the present invention may be straight, branched, or cyclic. In one preferred embodiment of the present invention, the vinyl acetate co-monomer is a compound having the structure of formula (IV) wherein R³ is methyl and R⁴ is hydrogen. It is understood to one skilled in the art that suitable vinyl acetate co-monomers also include any analogous vinyl acetates of the above-mentioned vinyl acetate co-monomers.

In one embodiment of the present invention, the copolymer comprises the following repeating unit: 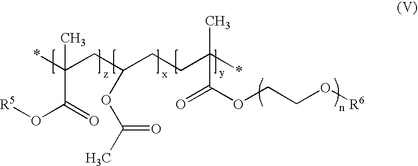
wherein R⁵ is an alkyl group having 1 to 12 carbon atoms; R⁶ is a hydrogen atom or an alkyl group having 1 to 6 carbon atoms; n is an integer of 2 to 100; and x, y and z are the same or different, and are independently an integer of 10 to 2500. Preferably, n is an integer of 2 to 10.

In one embodiment of the present invention, the copolymer comprises the following repeating unit: 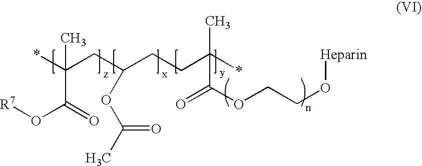
wherein R⁷ is an alkyl group of 1 to 12 carbons; n is an integer of 2 to 100; and x, y and z are the same of different, and are independently an integer of 10 to 2500. Preferably, n is an integer of 2 to 10. Heparin is a well-known anticoagulant used to decrease the clotting ability of the blood and prevent harmful clots from forming in the blood vessels. The attachment of heparin to the polyethylene oxide moiety enables the resulting copolymer to be soluble in common organic solvents used in coating processes, thereby eliminating the use of water and improving the coating's morphology. A coating comprising the copolymer of formula (VI) has enhanced hemocompatibility, thus is particularly useful as the coating for implantable medical devices.

It is preferable that the inventive copolymer has a tunable polymer molecular weight ranging from about 10K to about 5000K Daltons to enable the formation of a polymer with desirable mechanical durability and adequate adhesiveness. Since the mechanical durability of a coating improves upon increasing polymer molecular weight, it is especially preferable that the inventive copolymer has a high polymer molecular weight of 50K to 5000K Daltons for use in coatings for certain medical devices (e.g., stents) which require expansion and deployment in vivo.

The amphiphilic coating of the present invention may additionally include co-solvents and/or other additives to facilitate high quality film formation, such as plasticizers, antifoaming agents, anticrater agents, and coalescing solvents. Other suitable additives to the amphiphilic coating material include, but are not limited to: bioactive agents, antimicrobial agents, antithrombogenic agents, antibiotics, pigments, radiopacifiers and ion conductors. Details concerning the selection and amounts of such ingredients are known to those skilled in the art.

The inventive amphiphilic coating material may be applied to at least a portion of one surface of an article. In some embodiments, the inventive coating is applied to all exposed surfaces of an article. The thickness of the amphiphilic coating may vary depending on the process used in forming the coating as well as the intended use of the article. Typically, and for a medical device, the inventive coating is applied to a thickness from about 10 to about 5000 Å, with a thickness from about 20 to about 1000 Å being more typical.

When applied on at least a portion of one surface of an article, the hydrophobic backbone of the inventive copolymer forms a non-swellable base layer and adheres firmly to the underlying surface, while the hydrophilic pendent chains of the inventive copolymer hydrate and swell under physiological conditions and form a lubricious and hemocompatible surface. The low-friction and hemocompatibility of the hydrophilic pendent chains provide excellent anti-thrombotic properties that potentially reduce subacute thrombosis (SAT). Further, the hydrophobic backbone has a predefined molecular weight with a narrow range of distribution which improves the mechanical durability of the polymer, while the hydrophilic pendent chains are adjustable to various lengths to obtain the desirable elasticity of the polymer. Thus, the inventive coating is robust, i.e., mechanically durable, and flexible, i.e., elastic. The robustness and flexibility of the inventive polymer significantly reduce flaking, peeling, and other defects commonly seen in many current coatings on medical devices, particularly the coatings on stents. Accordingly, the present invention provides an improved biocompatible coating, which has not only inert hydrophilic surfaces to be in contact with body tissue of a mammal, for example, a human, sufficiently lubricious to reduce restenosis, or thrombosis, or other undesirable reactions, but also a hydrophobic backbone to firmly adhere to the underlying surface sufficiently durable to resist cracking when formed on an article, for example, a medical device.

The inventive amphiphilic coating may also be applied to control the elution of a therapeutic dosage of a pharmaceutical agent from a medical device base coating, for example, a stent base coating. The basecoat generally comprises a matrix of one or more drugs, agents, and/or compounds and a biocompatible material such as a polymer. The control over elution results from either a physical barrier, or a chemical barrier, or a combination thereof. The elution is controlled by varying the thickness of the coating, thereby changing the diffusion path length for the drugs, agents, and/or compounds to diffuse out of the basecoat matrix. Essentially, the drugs, agents and/or compounds in the basecoat matrix diffuse through the interstitial spaces in the coating. Accordingly, the thicker the coating, the longer the diffusion path, and conversely, the thinner the coating, the shorter the diffusion path. It is important to note that both the basecoat and the coating thickness may be limited by the desired overall profile of the article on which they are applied.

The properties of the inventive copolymer may be tuned via adjusting the molar ratios of the co-monomers. In other words, the molar ratios of the co-monomers may be adjusted according to the desired properties of the inventive copolymer. For example, when biologically active molecules are attached to the polyethylene oxide substituted methacrylates, the co-monomers are in a molar ratio that ensures desired mechanical strength of the copolymer while providing a hydrophilic environment for retaining the optimal activity of the biologically active molecules. Preferably, the copolymer has the alkyl methacrylate or alkyl acrylate, the vinyl acetate, and the polyethylene oxide substituted methacrylate in a mole ratio of 1:1:1.

The structure of the hydrophobic backbone and the molecular weight of the inventive polymer may be controlled through employment of various polymerization methods. The preferred polymerization methods of the present invention include group transfer polymerization (GTP), anionic polymerization, and living polymerization. The more preferred polymerization method of the present invention is GTP. GTP is a living polymerization technique which involves a Michael-type addition using a silyl ketene acetal initiator (see, for example, Vamvakaki, M. et al., Polymer, 40, 1999, 5161-5171). Many conventional polymerization methods require chemical crosslinking reactions, high temperature curing processes, and/or plasma treatments, which not only have very limited control over the polymer backbone structure and the molecular weight distribution, but also cause damage to the drug-content in the underlying basecoat. Unlike those conventional polymerization methods, GTP can be used for the synthesis of controlled structure acrylate or methacrylate polymers of narrow molecular weight distribution at ambient temperature. The hydrophilic pendent chains of the inventive copolymer provide desired lubricious properties and hemocompatibility, and the length of these hydrophilic pendent chains can be controlled via using monomers with desirable number of repeating ethylene oxide units in the polymerization reactions. The preferred monomers for the polymerization are the monomers that contain 2 to 10 repeating ethylene oxide units. Moreover, the polyethylene oxide moieties serve as functional pendant chains whereby one or more biologically active molecules may be introduced via conjugating processes that are compatible with the biologically active molecules.

A general co-polymerization process of the present invention is shown in Scheme 1 as below: 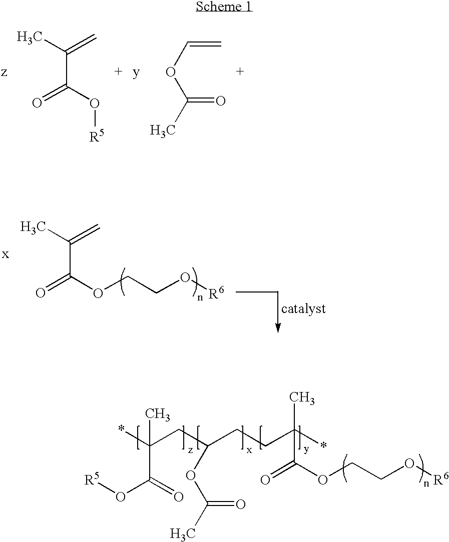
wherein R⁵ is an alkyl group having 1 to 12 carbon atoms; R⁶ is a hydrogen atom or an alkyl group having 1 to 6 carbon atoms, x, y, and z are independently an integer of 10 to 2500, and n is an integer of 2 to 100. Catalysts suitable for the above polymerization process are known to one skilled in the art. Examples of the catalysts include, but are not limited to: 1-methoxy-1-trimethylsiloxy-2-methyl-1-propene (MTS), n-tetrabutylammonium bibenzoate (TBABB), and other polymerization initiators.

A general conjugation process of the present invention is shown in Scheme 2 as below: 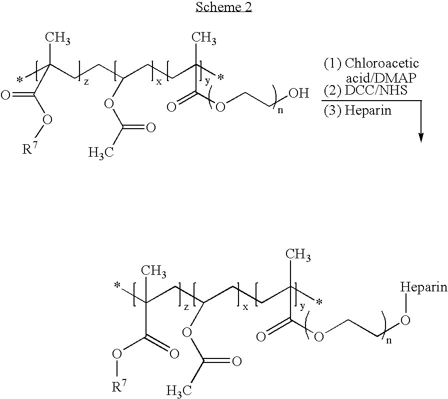
wherein R⁷ is an alkyl group having 1 to 12 carbon atoms, x, y, and z are independently an integer of 10 to 2500, and n is an integer of 2 to 100. In the conjugation process illustrated by Scheme 2, the polyethylene oxide moiety is activated by treating the hydroxyl group with chloroacetic acid in the presence of DMAP (dimethylaminopyridine) and then DCC (dicyclohexylcarbodiimide) and NHS(N-hydroxyl succinimide). Next, heparin is conjugated with the activated polyethylene oxide moiety. The stepwise activation of the copolymer rather than derivatizing heparin not only minimizes the undesirable crosslinking reactions commonly seen in the derivatization of heparin, but also provides a high degree of control over conjugation extent. Further, the conjugation process of Scheme 2 is conducted under mild conditions, thereby eliminating the undesirable long-lasting free radicals generated in conventional UV-radiating conjugation process.

The present invention also provides an article having the inventive amphiphilic coating thereon. The inventive amphiphilic coating is on at least a portion of one surface of the article. The at least a portion of one surface of the article may be a surface of a polymeric coat, a plastic substance, ceramic, steel, or other alloy metals. The article that may be coated with the inventive amphiphilic coating material may be in any shape, and is preferably a medical device or a component of a medical device. The term “medical device” as used herein denotes a physical item used in medical treatment, which includes both external medical devices and implantable medical devices. The medical devices that may be coated with the inventive amphiphilic coating material include, but are not limited to: catheters, guidewires, drug eluting stents, cochlear implants, retinal implants, gastric bands, neurostimulation devices, muscular stimulation devices, implantable drug delivery devices, intraocular devices, and various other medical devices.

The present amphiphilic coating material may be applied to the surface of an article using conventional coating techniques, such as, for example, spray coating, ultrasonic coating, dip coating, and the like. In a dip coating process, the article is immersed in a bath containing the amphiphilic coating material and then removed. A dwelling time ranging from about 1 minute to about 2 hours may be used depending of the material of construction, complexity of the device, and the desired coating thickness. Next, the article coated with the amphiphilic coating material may be allowed to dry to provide a dry coating. Drying may be accomplished merely by standing at ambient conditions or may be accelerated by heating at mild temperatures, such as about 30° C. to about 65° C.

While the present invention has been particularly shown and described with respect to preferred embodiments thereof, it will be understood by those skilled in the art that the foregoing and other changes in forms. And details may be made without departing from the spirit and scope of the invention. It is therefore intended that the present invention not be limited to the exact forms and details described and illustrated but fall within the scope of the appended claims.

Having thus described in our invention in detail, what we claim as new and desire to secure by the Letters Patent is:

1. An amphiphilic coating material for applying on at least a portion of one surface of an article, said amphiphilic coating material comprising a copolymer containing one or more alkyl methacrylate or alkyl acrylate co-monomer units; one or more vinyl acetate co-monomer units; and up to 40% mole of polyethylene oxide substituted methacrylate co-monomer units.

2. The amphiphilic coating material of claim 1, wherein the polyethylene oxide substituted methacrylate co-monomer unit comprises the following structure: 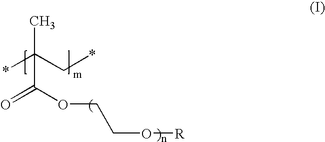
wherein R is a hydrogen atom, an alkyl group of 1 to 6 carbon atoms, or a biologically active molecule; n is an integer of 2 to 100; and m is an integer of 100 to 5000.

3. The amphiphilic coating material of claim 1, wherein n is 2 to 10.

4. The amphiphilic coating material of claim 1, wherein said amphiphilic coating material has a thickness of about 10 to about 5000 Å.

5. The amphiphilic coating material of claim 1, wherein the copolymer has a tunable molecular weight ranging from about 10K to about 5000K Daltons.

6. The amphiphilic coating material of claim 2, wherein the biologically active molecule is an anti-thrombogenic agent, an immuno-suppressant agent, an anti-neoplastic agent, an anti-inflammatory agent, an angiogenesis inhibitor, or a protein kinase inhibitor.

7. The amphiphilic coating material of claim 2, wherein the biologically active molecule is selected from the group consisting of heparin, rapamycin, paclitaxel, pimecrolimus, and the analogs and derivatives thereof.

8. The amphiphilic coating material of claim 1, wherein the one or more methacrylate co-monomers are selected from the group consisting of methyl methacrylate, ethyl methacrylate, butyl methacrylate, hexyl methacrylate, and octyl methacrylate.

9. The amphiphilic coating material of claim 1, wherein the one or more acrylate co-monomers are selected from the group consisting of methyl acrylate, ethyl acrylate, butyl acrylate, hexyl acrylate, and octyl acrylate.

10. The amphiphilic coating material of claim 1, wherein the copolymer comprises the following repeating unit: 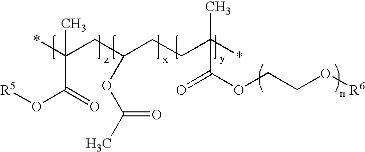
wherein R⁵ is an alkyl group having 1 to 12 carbon atoms; R⁶ is a hydrogen atom or an alkyl group having 1 to 6 carbon atoms; n is an integer of 2 to 100; and x, y and z are the same or different, and are independently an integer of 10 to 2500.

11. The amphiphilic coating material of claim 1, wherein the copolymer comprises the following repeating unit: 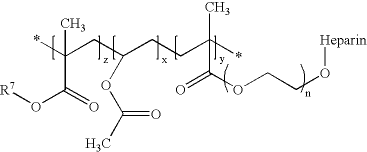
wherein R⁷ is an alkyl group of 1 to 12 carbons; n is an integer of 2 to 100; and x, y and z are the same of different, and are independently an integer of 10 to 2500.

12. An amphiphilic coating material for applying on at least one surface of an article, said amphiphilic coating material comprising a copolymer containing one or more alkyl methacrylate or alkyl acrylate co-monomer units; one or more vinyl acetate co-monomer units; and up to 40% mole of polyethylene oxide substituted methacrylate co-monomer units, wherein one or more biologically active molecules are covalently bonded to the polyethylene oxide substituted methacrylate co-monomer units.

13. An article having an amphiphilic coating thereon, said amphiphilic coating comprising a copolymer containing one or more alkyl methacrylate or alkyl acrylate co-monomer units; one or more vinyl acetate co-monomer units; and up to 40% mole of polyethylene oxide substituted methacrylate co-monomer units.

14. The article of claim 13, wherein the polyethylene oxide substituted methacrylate co-monomer unit comprising the following structure: 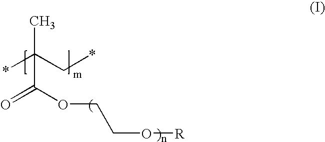
wherein R is a hydrogen atom, an alkyl group of 1 to 6 carbon atoms, or a biologically active molecule; n is an integer of 2 to 100; and m is an integer of 10 to 500.

15. The article of claim 14, wherein n is an integer of 2 to 10.

16. The article of claim 14, wherein the biologically active molecule is an anti-thrombogenic agent, an immuno-suppressant agent, an anti-neoplastic agent, an anti-inflammatory agent, an angiogenesis inhibitor, or a protein kinase inhibitor.

17. The article of claim 14, wherein the biologically active molecule is selected from the group consisting of heparin, rapamycin, paclitaxel, pimecrolimus, and the analogs and derivatives thereof.

18. The article of claim 13, wherein the amphiphilic coating has a thickness of about 10 to about 5000 Å.

19. The article of claim 13, wherein the copolymer has a tunable molecular weight ranging from about 10K to about 500K Daltons.

20. The article of claim 13, wherein the copolymer comprises the following repeating unit: 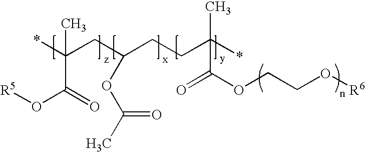
wherein R⁵ is an alkyl group having 1 to 12 carbon atoms; R⁶ is a hydrogen atom or an alkyl group having 1 to 6 carbon atoms; n is an integer of 2 to 100; and x, y and z are the same or different, and are independently an integer of 10 to 5000.

21. The article of claim 13, further comprising one or more biologically active molecules covalently bonded to the polyethylene oxide substituted methacrylate co-monomer units.

22. The article of claim 21, wherein the copolymer comprises the following repeating unit: 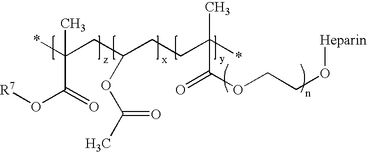
wherein R⁷ is a hydrogen atom or an alkyl group of 1 to 6 carbons; n is an integer of 2 to 100; and x, y and z are the same of different, and are independently an integer of 10 to 2500.

23. The article of claim 13 is a medical device or a component of a medical device.
